(19) United States
(12) Patent Application Publication
(10) Pub. No.: US 2008/0091075 A1
(43) Pub. Date: Apr. 17, 2008

(54) FLEXIBLE FIBEROPTIC ENDOSCOPE WITH INTRODUCER

(75) Inventor: Seckin Ulualp, Dallas, TX (US)

Correspondence Address: ROBERT W STROZIER, P.L.L.C, PO BOX 429, BELLAIRE, TX, 77402-0429, US

(73) Assignee: The Board of Regents of The University of Texas System, Austin, TX (US)

(21) Appl. No.: 11/870,957

(22) Filed: Oct. 11, 2007

Related U.S. Application Data
(60) Provisional application No. 60/851,034, filed on Oct. 11, 2006.

(51) Int. Cl. A61B 1/06

(52) U.S. Cl. 600/178

(57) ABSTRACT

A new endoscope apparatus is disclosed that includes a probe having an openable compartment housing a deployable medical apparatus, where the deployable medical apparatus can be deployed after the endoscope is properly positioned within an animal including a human and methods for making and using same.

### RELATED APPLICATIONS

This application claims priority to U.S. Provisional Patent Application Ser. No. 60/851,034, filed 11 Oct. 2006 (Oct. 11, 2006 or Nov. 10, 2006).

### BACKGROUND OF THE INVENTION

1. Field of the Invention

The present invention relates to a flexible fiberoptic endoscope assisted instrumentation (FFEAI) apparatus for esophageal and pharyngeal pH monitoring and multichannel intraluminal impedance monitoring and method for making and using same.

More particularly, the present invention relates to a flexible fiberoptic endoscope assisted instrumentation (FFEAI) apparatus including (a) a TV monitor; (b) a camera, (c) a light source, (d) a VHS or digital recording system, (e) a printer, (f) a flexible fiberoptic endoscope with introducer and (g) a medical device to be introduced via the introducer.

2. Description of the Related Art

The reflux of gastric contents into the esophagus, i.e., gastroesophageal reflux (GER), and pharynx, i.e., laryngopharyngeal reflux (LPR), have been implicated in the pathogenesis of a wide variety of aerodigestive tract disorders in adults, adolescents, children, and infants. Otolaryngologists, pulmonologists, gastroenterologists, and pediatricians are involved in the management of LPR induced airway disorders. It is estimated that 4% to 10% otolaryngologic signs and symptoms are associated with LPR. Patients with GER commonly report typical symptoms of GERD such as heartburn, acid regurgitation, and water brash. On the other hand, patients suffering from LPR associated airway disorders experience the typical symptoms of GER infrequently. Chronic intermittent nature of the LPR induced symptoms makes the diagnosis of LPR induced airway disorders challenging. Therefore, the efficient management of patients with LPR associated airway disorders including but not limited to otolaryngologic disorders require a meticulous synthesis of the information obtained from a complete otolaryngologic examination, diagnostic tests, and response to treatment.

Significance

A detailed history and laryngeal examination are essential components of complete otolaryngologic examination of the patients with LPR. Laryngeal examination is performed using indirect laryngoscopy, fiberoptic laryngoscopy, or direct laryngoscopy. Flexible fiberoptic laryngoscopy is commonly used in the office-based examination of the larynx. Also, laryngeal signs do not always correlate well with the diagnostic tests such as barium esophagram, gastric scintiscan, and esophagopharyngeal pH monitoring. Currently, esophagopharyngeal pH monitoring is considered as the gold standard method for detecting the GER and LPR. Esophagopharyngeal pH monitoring is performed by placing a catheter with single or multiple recording site into the esophagus and pharynx. A pocket sized computer records acid reflux in the esophagus and the pharynx during the study period reaching up to 24 hour. Placement of a probe in the laryngopharyngeal region during esophagopharyngeal pH monitoring is considered essential in detecting the acid reflux exposure in the laryngopharyngeal region.

Current Diagnostic Techniques and Challenges

Traditionally, pharyngeal pH monitoring site is determined based on the manometrically determined location of the upper border of the upper esophageal sphincter. Manometric assessment of upper and lower esophageal sphincters requires passing catheter thru the nose into the esophagus. The procedure may take up to 30 minutes and may cause some discomfort to the patients. Manometrically determined location of pharyngeal pI-I monitoring is commonly used in adult patients. In children, the use of pharyngeal pH monitoring has been limited due to technical and practical difficulties to determine the location for esophageal and pharyngeal pH monitoring. Placement of pH probe under fluoroscopic guidance, radiologic confirmation of pH probe location, and estimated probe location based on the body length have been used to conduct esophageal and pharyngeal pH monitoring in infants and children. However, the techniques used in children have disadvantages of exposing a child to radiation and variation in the location pharyngeal pH probe. Development of new technologies relatively easy and fast to minimize discomfort, eliminating the need for esophagopharyngeal manometry and exposure to radiation and providing a consistent pharyngeal pH monitoring site which is needed to improve pharyngeal pH monitoring in adults and children suffering from laryngopharyngeal reflux.

Flexible fiberoptic endoscopes are commonly used to assess the airway and the esophagus. In the late 1990's, pH probe placement in the upper esophageal sphincter under the view of flexible fiberoptic laryngoscope was reported in adults. Nevertheless, this technique suffers from the need to pass flexible fiberoptic endoscope and pH catheter separately, thus, causing more discomfort and longer duration of procedure and increased risk for tissue damage in addition to being a two person procedure. The present invention overcomes the technical and practical difficulties of the existing methodologies in esophageal and pharyngeal pH monitoring and multichannel intraluminal impedance monitoring.

### SUMMARY OF THE INVENTION

The present invention provides a flexible fiberoptic endoscope assisted instrumentation (FFEAI) apparatus for esophageal and pharyngeal pH monitoring and multichannel intraluminal impedance monitoring.

The present invention also provides a endoscope apparatus including a probe having an openable compartment including a deployable medical apparatus and a light emitting and light receiving component. The apparatus also includes a monitoring system including an imaging component and an image processing component.

The present invention also provides a endoscope apparatus including (a) a TV monitor; (b) a camera, (c) a light source, (d) a VHS or digital recording system, (e) a printer, (f) a flexible fiberoptic endoscope with introducer, and (g) a deployable medical device.

The present invention also provides a flexible fiberoptic apparatus including a fiberoptic light delivery system, a fiberoptic light receiving system and a probe having a proximal end and a distal end. The fiberoptic light delivery system includes one or a plurality of optical fibers having distal ends that terminates in the distal end of the probe and proximal ends terminating adjacent a light source. The fiberoptic light receiving system including one or a plurality of optical fibers having proximal ends that terminate in the distal end of the probe and distal ends that terminate adjacent a detector such as a camera. The probe includes an openable compartment housing a medical apparatus such as a catheter, monitoring device or sensing device. In the case where the medical apparatus requires a wire, then the wire is connect to the medical apparatus at its proximal end and to a detector at its distal end. The light delivery system delivers light from the light source through the optical fibers out the distal end of the probe to illuminate an area of interest. The light receiving system receives light reflected from the area of interest, which can be received by a camera, optionally processed and displayed on an output device so the probe can be positioned adjacent a deployment site. The openable compartment can then be opened and the medical apparatus deployed. After deployment, all non-essential equipment is removed the endoscope and fiberoptic systems are removed leaving the medical device and its wire is needed. Of course, for wireless medical apparatuses, the entire device save the medical apparatus is removed.

The present invention provides a method for deploying a medical apparatus in an upper GI track, respiratory track or any other site of an animal including a human accessible using an endoscope, where the method includes the step of threading an endoscope probe attached to a cable connected to an image capture and analyzing unit adjacent through an esophagus to a site in the upper GI track, respiratory track or any other site of an animal including a human accessible using an endoscope. Once the probe is properly positioned, a slidable member is transitioned from its closed state to its opened state opening a compartment in the probe housing a deployable medical apparatus. After the slidable member is transitioned, the deployable medical apparatus is deployed. After deployment, the endoscope probe and non-essential cables are withdrawn leaving the deployable medical apparatus, and if the deployable medical apparatus is wireless, then no cables remain deployed.

### BRIEF DESCRIPTION OF THE DRAWINGS

The invention can be better understood with reference to the following detailed description together with the appended illustrative drawings in which like elements are numbered the same:

FIGS. 1A&B depict an embodiment of an endoscope of this invention including a wired medical apparatus; and

FIGS. 2A&B depict another embodiment of an endoscope of this invention including a wireless medical apparatus.

### DETAILED DESCRIPTION OF THE INVENTION

The inventor has found that a flexible fiberoptic endoscope apparatus can be constructed with an openable compartment housing a catheter, a probe, an instrument or any other type of medical sensing device adjacent an area of interest in an animal to be monitored by the catheter, probe, instrument or other medical sensing device.

The present apparatus has numerous potential applications in the field of medicine and surgery. Immediate applications of the device include, but are not limited to, esophagopharyngeal pH monitoring and esophagopharyngeal multichannel intraluminal impedance monitoring. The apparatus can also be used as a regular flexible fiberoptic endoscope and as an introducer when needed. Potential applications encompass addressing the need to place a catheter or any device/agent such as radioactive seeds, chemotherapeutic agents, stents into the organs accessible via an endoscope in addition to all current indications and uses for flexible fiberoptic endoscope.

The following is the schematic representation of the concept of flexible fiberoptic endoscope with introducer of this invention. The introducer portion of the apparatus is similar to a port/channel of existing flexible fiberoptic endoscopes; however, the introducer has the capability of releasing an catheter or any other type of medical instrument by providing an open lumen. Once the lumen is opened, the catheter or any other type of medical device can be anchored to or simply released adjacent a desired location or site. Then, the flexible fiberoptic endoscope is removed leaving the catheter any other type of medical device behind.

The apparatus is well suited for use in children and adults by otolaryngologists, gastroenterologists, pulmonologists, pediatricians, general surgeons, and speech language pathologists.

The apparatus of this invention will permit the deployment of pH monitoring or impedance monitoring catheters with significantly reduced discomfort to patients. This apparatus eliminates the need to separately perform passing an endoscope and a catheter through the patient's nose. By minimizing the instrumentation having to be passed through the patient's nose, the risk of complications such as nosebleeds and trauma to the structures upper airway and esophagus is also significantly reduced. Furthermore, the apparatus and methods for using the apparatus have the potential to establish a reliable, safe method of esophagopharyngeal pH monitoring or multichannel intraluminal impedance monitoring in infants. Currently applied techniques in infants require estimation of a suitable location of a critical structure based on predetermined equations and X-ray confirmation of the location of the probes. With the apparatus of this invention, there is no need for confirming probe placement by X-ray and uniform probe placement can be achieved. Uniform probe placement among patients can potentially facilitate the establishment of normative diagnostic values.

The present flexible fiberoptic endoscope apparatus including an introducer is a novel instrument. The flexible fiberoptic endoscope apparatus with introducer includes a flexible fiberoptic endoscope with an incorporated introducer system. Incorporation of a special port into a flexible fiberoptic endoscope is well known. Flexible fiberoptic endoscopes with built in channel/port exist and are commonly used. Currently, flexible fiberoptic endoscopes with channels used, for various purposes such as suction, irrigation, biopsy, etc. are available. However, the channel/port used in the existing flexible fiberoptic endoscopes is not conducive to placing an esophageal or pharyngeal pH monitoring catheter as well as a multichannel intraluminal impedance monitoring catheter. Age appropriate sized systems are designed to have a port, which allows placement of release of a catheter or any other medical device. The port is designed to hold the medical device. The flexible fiberoptic with introducer has a system to release the medical device under direct view when the desired location is reached. Once the medical device is released, the flexible fiberoptic endoscope with introducer is removed.

Purpose

To provide a safe, reliable, practical, multipurpose flexible fiberoptic endoscope with the capability of placing a medical device at a desired location in an animal, where the endoscope can then be removed leaving behind the medical device.

Immediate Applications

The device described herein has numerous potential applications. Immediate applications of the device include, but are not limited to, esophagopharyngeal pH monitoring and esophagopharyngeal multichannel intraluminal impedance monitoring.

Currently, there is no flexible fiberoptic endoscope capable of delivery a medical device to a location in an animal including a human, infant or adult. The device of this invention will be beneficial for patients, physicians, and insurance companies. By using the proposed device for pH monitoring or impedance monitoring catheters, the discomfort level of the patients during the placement of the catheters will be significantly reduced. The apparatus of this invention will eliminate the need to perform esophagopharyngeal manometry, which involves passing a catheter through the nose, without first passing an endoscope through the nose prior to passing the catheter through the nose. By minimizing the instrumentation being passed through the nose, the risk of complications such as nosebleed and trauma to the structures upper airway and esophagus is also significantly reduced in addition to the diminished discomfort level of a patient. Furthermore, the apparatus and method of this invention has the potential to establish a reliable, safe method of esophagopharyngeal pH monitoring or multichannel intraluminal impedance monitoring in infants. Currently applied techniques in infants require estimation of the location of the critical structures based on predetermined equations and x-ray confirmation of the location of the probes. The present apparatus and method does not need confirmation of probe location using X-ray and uniform probe location is achieved. Uniform probe location among patients facilitates accurate diagnosis, monitoring of treatment, and establishment of normative values.

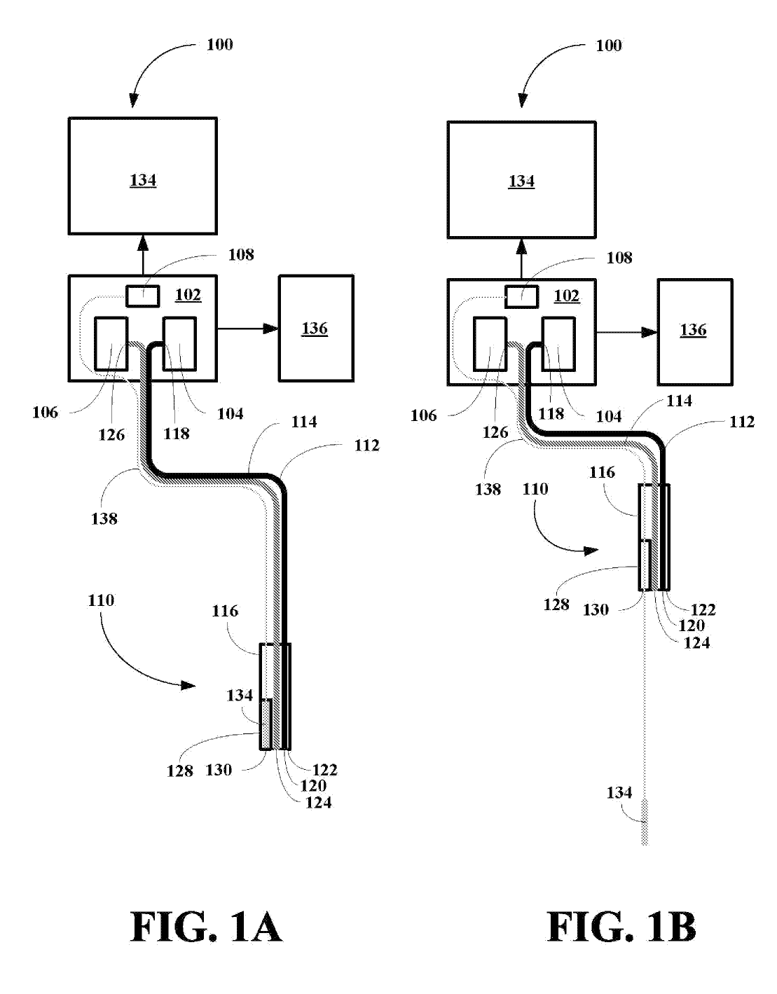

Referring now to FIGS. 1A&B, an embodiment of an endoscope apparatus of this invention, generally 100, is shown to include an imaging unit 102 having a light source 104, a light detector 106, and a medical apparatus detector 108. The apparatus 100 also includes an endoscope 110 having a fiberoptic light delivery system 112, a fiberoptic light receiving system 114 and a probe 116. The fiberoptic light delivery system 112 has a proximal end 118 that terminates at the light source 104 and a distal end 120 that terminates in a distal end 122 of the probe 116. The fiberoptic light receiving system 114 has a proximal end 124 that terminates in the distal end 112 of the probe 116 and a distal end 126 that terminates at the light detector 106. The probe 116 also includes a compartment 128 having an openable door 130 and housing a deployable medical apparatus 132. The apparatus 100 also includes a visual output device 134 and a hardcopy output device 136. The deployable medical apparatus 132 is connected to the medical apparatus detector 106 via a communication and/or power cable 138. FIG. 1B shows the endoscope after medical apparatus deployment and endoscope retraction.

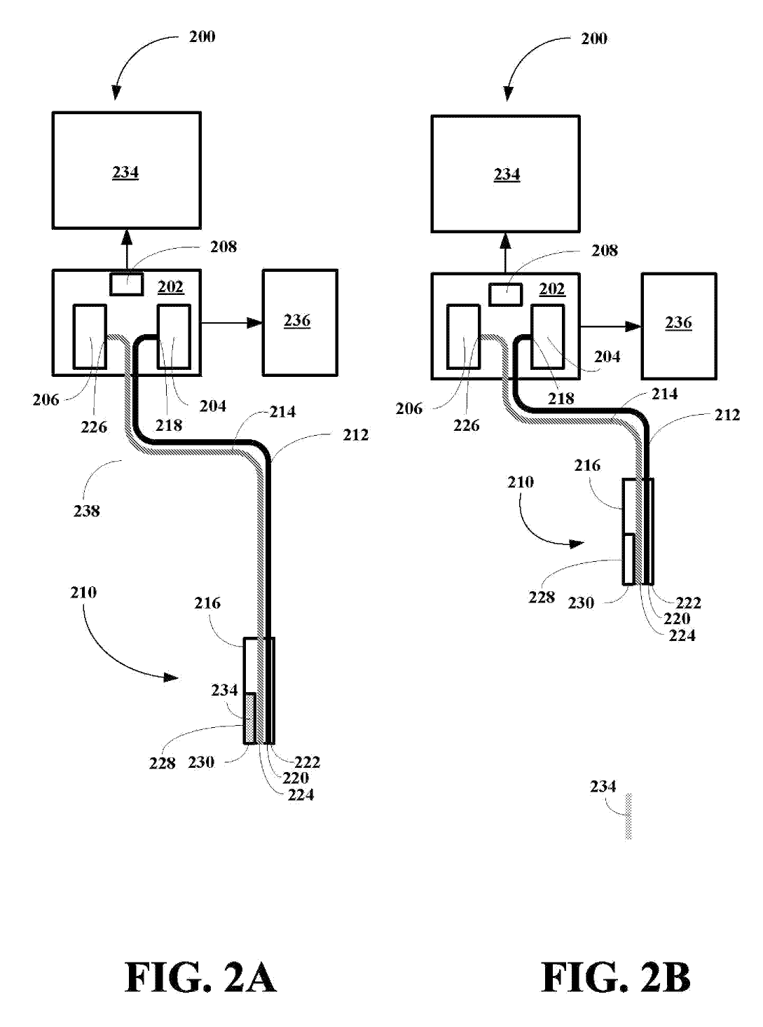

Referring now to FIGS. 2A&B, a, generally 200, is shown to include an imaging unit 202 having a light source 204, a light detector 206, and a medical apparatus detector 208. The apparatus 200 also includes an endoscope 210 having a fiberoptic light delivery system 212, a fiberoptic light receiving system 214 and a probe 216. The fiberoptic light delivery system 212 has a proximal end 218 that terminates at the light source 204 and a distal end 220 that terminates in a distal end 222 of the probe 216. The fiberoptic light receiving system 214 has a proximal end 224 that terminates in the distal end 212 of the probe 216 and a distal end 226 that terminates at the light detector 206. The probe 216 also includes a compartment 228 having an openable door 230 and housing a deployable medical apparatus 232. The apparatus 200 also includes a visual output device 234 and a hardcopy output device 236. The deployable medical apparatus 232 is a wireless apparatus that communications with the medical apparatus detector 208 via a wireless communication protocol. FIG. 2B shows the endoscope after medical apparatus deployment and endoscope retraction.

All references cited herein are incorporated by reference. Although the invention has been disclosed with reference to its preferred embodiments, from reading this description those of skill in the art may appreciate changes and modification that may be made which do not depart from the scope and spirit of the invention as described above and claimed hereafter.

1. An endoscope apparatus comprising:
a probe including an openable compartment housing a deployable medical apparatus,
a light emitting component and
light receiving component,
where the light emitting component is adapted to illuminate a travel path of the endoscope apparatus in an animal including a human, the light receiving component is adapted to receive light reflected from the path and where the probe is adapted to release the deployable medical apparatus at a desired an area of interest of the animal.

2. The apparatus of claim 1, further comprising:
a monitoring system including an imaging component and an image processing component adapted to receive light from the receiving component, capturing images and processing the images for monitoring.

3. The apparatus of claim 1, wherein the monitoring system further includes a TV monitor; a VHS or digital recording system, and a printer.

4. An endoscope apparatus comprising:
a flexible fiberoptic endoscope including:
a probe comprising:
an introducer having a deployable medical device housing within the introducer,
a light emitting component,
a light receiving component,
a monitoring system including an imaging component and an image processing component adapted to receive light from the light receiving component, to capture images from the received light and to process the images probe for monitoring,
where the light emitting component is adapted to illuminate a travel path of the endoscope apparatus in an animal including a human, the light receiving component is adapted to receive light reflected from the path and where the probe is adapted to release the deployable medical apparatus at a desired an area of interest of the animal.

5. A flexible fiberoptic apparatus comprising:
a probe including a proximal end, a distal end and an openable compartment housing a medical apparatus,
a light source,
a fiberoptic light delivery system including an optical fiber or a plurality of optical fibers having distal ends that terminate in the distal end of the probe and proximal ends that terminate adjacent a light source,
a fiberoptic light receiving system including an optical fiber or a plurality of optical fibers having proximal ends that terminate in the distal end of the probe and distal ends that terminate adjacent a monitoring system,
the monitoring system including the imaging component and an image processing component adapted to receive light from the receiving component, capturing images and processing the images for monitoring the probe as it passes into an animal including a human,
where the light emitting component is adapted to illuminate a travel path of the endoscope apparatus in an animal including a human, the light receiving component is adapted to receive light reflected from the path and where the probe is adapted to release the deployable medical apparatus at a desired an area of interest of the animal.

6. The apparatus of claim 1, wherein the medical apparatus includes a wire connected to the medical apparatus at its proximal end and to the monitoring system at its distal end and where the monitoring system includes a component for monitoring output from the medical apparatus.

7. A method for deploying a medical apparatus in an animal comprising the steps of:
threading an endoscope into an animal including a human, where the endoscope comprises:
a probe including a proximal end, a distal end and an openable compartment housing a deployable medical apparatus and having a slidable member adapted to open and close the openable compartment,
a fiberoptic light delivery system including an optical fiber or a plurality of optical fibers having distal ends that terminate in the distal end of the probe and proximal ends that terminate adjacent a light source,
a fiberoptic light receiving system including an optical fiber or a plurality of optical fibers having proximal ends that terminate in the distal end of the probe and distal ends that terminate adjacent a monitoring system,
where the probe attached to a cable connected to a monitoring system,
passing light through the fiberoptic light delivery system from the light source to illuminate a path of the probe during the threading step,
converting the light received from the fiberoptic light receiving system and into images in the monitoring system during the threading step,
monitoring a location of the probe from the images produced by the monitoring system during the threading step,
positioning the probe at an area of interest in the animal based on the monitoring
after the probe is properly positioned adjacent the area, sliding the slidable of the probe to open the openable compartment,
deploying the deployable medical apparatus adjacent the area n,
withdrawing the endoscope leaving the deployable medical apparatus in place, and
monitoring output from the deployable medical after deployment.

8. The method of claim 7, wherein the deployable medical apparatus includes a wire connected to the deployable medical apparatus.

9. The method of claim 8, further comprising the step of:
removing the deployable medical apparatus via the wire.

10. The method of claim 7, wherein the deployable medical apparatus is a wireless deployable medical apparatus.
